(19) United States
(12) Patent Application Publication
(10) Pub. No.: US 2002/0040011 A1
(43) Pub. Date: Apr. 4, 2002

(54) Naphthoquinone compositions and uses thereof

(76) Inventor: Dennis M. Brown, Menlo Park, CA (US)

Correspondence Address: FLEHR HOHBACH TEST, ALBRITTON & HERBERT LLP, 4 Embarcadero Center - Suite 3400, San Francisco, CA, 94111-4187, US

(21) Appl. No.: 09/872,760

(22) Filed: May 31, 2001

Related U.S. Application Data
(60) Provisional application No. 60/208,645, filed on Jun. 1, 2000.

(51) Int. Cl. A61K 31/7076; A61K 31/7064; A61K 31/122; A61K 33/24

(52) U.S. Cl. 514/45; 514/46; 514/49; 514/682; 424/649

(57) ABSTRACT

A method of treatment of a host with a cellular proliferative disease, comprising contacting the host with a naphthoquinone and an antiproliferative agent, each in an amount sufficient to modulate said cellular proliferative disease, is described. In some embodiments, the naphthoquinone comprises menadione (Kativ-G; 2-Methyl-1,4-Naphthoquinone; Menaphthone; Vitamin K3; Panosine; 2-Methyl-1,4-naphthalenedione; Vitamin K2(0); Methyl-1,4-naphthalenedione; Methyl-1,4-naphthoquinone). Antiproliferative agents of the invention comprise alkylating agents, intercalating agents, metal coordination complexes, pyrimidine nucleosides, purine nucleosides, inhibitors of nucleic acid associated enzymes and proteins, and agents affecting structural proteins and cytoplasmic enzymes. The invention comprises the described methods as well as compositions comprising a naphthoquinine and an antiproliferative agent.

This application claims the benefit of U.S. Provisional Application No. 60/208,645, filed Jun. 1, 2000.

### FIELD OF THE INVENTION

The technical field of the invention is the use of naphthoquinones with antiproliferative agents to treat a host with a cellular proliferative disease.

### BACKGROUND OF THE INVENTION

There is considerable interest in modulating the efficacy of currently used antiproliferative agents to increase the rates and duration of antitumor effects associated with conventional antineoplastic agents.

Conventional antiproliferative agents used in the treatment of cancer are broadly grouped as chemical compounds which (1) affect the integrity of nucleic acid polymers by binding, alkylating, inducing strand breaks, intercalating between base pairs or affecting enzymes which maintain the integrity and function of DNA and RNA; (2) chemical agents that bind to proteins to inhibit enzymatic action (e.g. antimetabolites) or the function of structural proteins necessary for cellular integrity (e.g. antitubulin agents). Other chemical compounds that have been identified to be useful in the treatment of some cancers include drugs which block steroid hormone action for the treatment of breast and prostate cancer, photochemically activated agents, radiation sensitizers and protectors.

Of special interest to this invention are those compounds that directly affect the integrity of the genetic structure of the cancer cells. Nucleic acid polymers such as DNA and RNA are prime targets for anticancer drugs. Alkylating agents such as nitrogen mustards, nitrosoureas, aziridine containing compounds directly attack DNA. Metal coordination compounds such as cisplatin and carboplatin similarly directly attack the nucleic acid structure resulting in lesions that are difficult for the cells to repair which, in turn, can result in cell death. Other nucleic acid affecting compounds include anthracycline molecules such as doxorubicin, which intercalates between the nucleic acid base pairs of DNA polymers, bleomycin which causes nucleic acid strand breaks, fraudulent nucleosides such as pyrimidine and purine nucleoside analogs which are inappropriately incorporated into nucleic polymer structures and ultimately cause premature DNA chain termination. Certain enzymes that affect the integrity and functionality of the genome can also be inhibited in cancer cells by specific chemical agents and result in cancer cell death. These include enzymes that affect ribonucleotide reductase (e.g. hydroxyurea, gemcitabine), topoisomerase I (e.g. camptothecin) and topoisomerase II (e.g. etoposide).

One of the most broadly used of these DNA targeted anticancer drugs is cisplatin (cis-diamminedichloroplatinum II). This compound is active against several human cancers including testicular, small-cell lung, bladder, cervical and head and neck cancer.

While the clinical activity of cisplatin against these forms of cancers are demonstratable, improvements in tumor response rates, duration of response and ultimately patient survival are still sought. The invention described herein demonstrates the novel use of the naphthoquinones and derivatives including menadione which can potentiate the antitumor effects of chemotherapeutic drugs, in particular, cisplatin.

### SUMMARY OF THE INVENTION

Methods and compositions are provided for the treatment of a host with a cellular proliferative disease, particularly a neoplasia. In the subject methods, pharmaceutically acceptable naphthoquinone and an antiproliferative agent are administered in an amount sufficient to modulate the cellular proliferative disease.

### DETAILED DESCRIPTION OF THE FIGURES

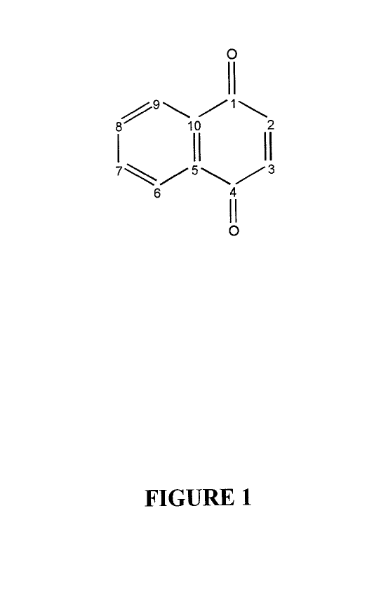

FIG. 1 depicts the general structure of a naphthoquinone.

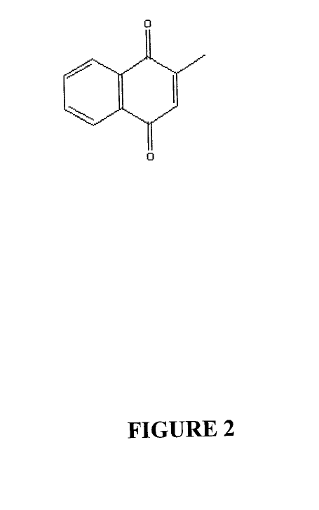

FIG. 2 depicts the general structure of the naphthoquinone analog menadione.

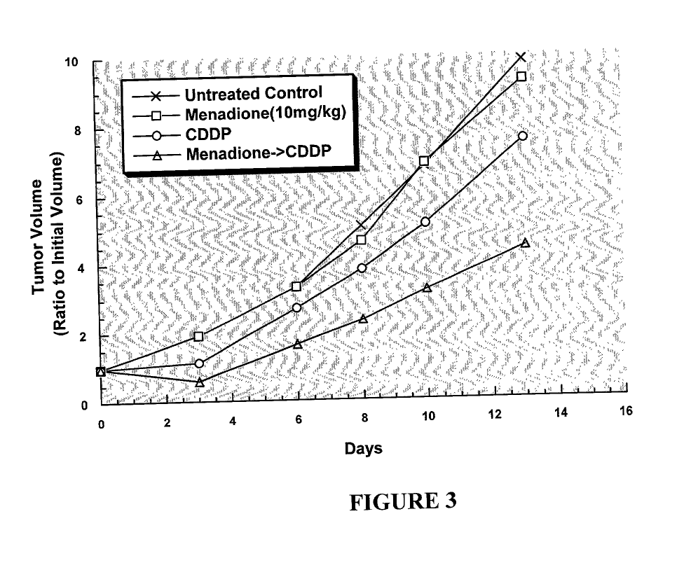

FIG. 3 shows tumor growth delay, as tumor volume on days after treatment with the naphthoquinone analog, menadione, with cisplatin (CDDP), or with menadione followed by cisplatin.

### DETAILED DESCRIPTION OF THE INVENTION

Methods and compositions are provided for the treatment of a host with a cellular proliferative disease, particularly a neoplasia. In the subject methods, a pharmaceutically acceptable naphthoquinone is administered, preferably systemically, in conjunction with an antiproliferative agent to improve the anticancer effects. In a preferred embodiment, the naphthoquinone provides a chemopotentiator effect.

The agents are provided in amounts sufficient to modulate a cellular proliferative disease. In one embodiment, modulation of a cellular proliferative disease comprises a reduction in tumor growth. In another embodiment, modulation of a disease comprises inhibition of tumor growth. In another embodiment, modulation of a cellular proliferative disease comprises an increase in tumor volume quadrupling time (described below). In another embodiment, modulation of a cellular proliferative disease comprises a chemopotentiator effect. In another embodiment, modulation of a disease comprises a chemosensitizing effect. In other embodiments, modulation of a disease comprises cytostasis. In still other embodiments, modulation of a disease comprises a cytotoxic effect.

A chemical agent is a “chemopotentiator” when it enhances the effect of a known antiproliferative drug in a more than additive fashion relative to the activity of the chemopotentiator or antiproliferative agent used alone. In some cases, a “chemosensitizing” effect may be observed. This is defined as the effect of use of an agent that if used alone would not demonstrate significant antitumor effects but would improve the antitumor effects of an antiproliferative agent in a more than additive fashion than the use of the antiproliferative agent by itself.

As used herein, the term “naphthoquinone” includes all members of that chemical family including menadione and analogs thereof. The naphthoquinone family is defined by chemical structure as depicted in FIG. 1.

A naphthoquinone analog is further defined but not limited to substituent changes at carbons 2, 3, 6, 7, 8, or 9 of the structure shown in FIG. 1. Examples substituent changes at carbons 2, 3, 6, 7, 8 and 9 include nitro (NO₂), alkyl nitro, amino (NH₂), alkyl amino, carboxamide, alkyl carboxamide, alkyl with carbon chain length of from one to five carbons (C₁₋₅), alkoxy with carbon chain length of from one to five carbons (OC₁₋₅), hydroxyl, and hydrogen. In a preferred embodiment, a naphthoquinone analog has the structure of menadione, shown in FIG. 2.

A specific example of a naphthoquinone is menadione which is also known by the following chemical synonyms: Kativ-G; 2-Methyl-1,4-Naphthoquinone; Menaphthone; Vitamin K3; Panosine; 2-Methyl-1,4-naphthalenedione; Vitamin K2(0); Methyl-1,4-naphthalenedione; Methyl-1,4-naphthoquinone (FIG. 2), including salt forms such as menadione sodium bisulfite and menadiol sodium diphosphate.

As used herein, antiproliferative agents are compounds which induce cytostasis or cytotoxicity. “Cytostasis” is the inhibition of cells from growing, while “cytotoxicity” is defined as the killing of cells. Specific examples of antiproliferative agents include: antimetabolites, such as methotrexate, 5-fluorouracil, gemcitabine, cytarabine, pentostatin, 6-mercaptopurine, 6-thioguanine, L-asparaginase, hydroxyurea, N-phosphonoacetyl-L-aspartate (PALA), fludarabine, 2-chlorodeoxyadenosine, and floxuridine; structural protein agents, such as the vinca alkaloids, including vinblastine, vincristine, vindesine, vinorelbine, paclitaxel, and colchicine; agents that affect NF-κB, such as curcumin and parthenolide; agents that affect protein synthesis, such as homoharringtonine; antibiotics, such as dactinomycin, daunorubicin, doxorubicin, idarubicin, bleomycins, plicamycin, and mitomycin; hormone antagonists, such as tamoxifen and luteinizing hormone releasing hormone (LHRH) analogs; nucleic acid damaging agents such as the alkylating agents mechlorethamine, cyclophosphamide, ifosfamide, chlorambucil, dacarbazine, methylnitrosourea, semustine (methyl-CCNU), chlorozotocin, busulfan, procarbazine, melphalan, carmustine (BCNU), lomustine (CCNU), and thiotepa, the intercalating agents doxorubicin, dactinomycin, daurorubicin and mitoxantrone, the topoisomerase inhibitors etoposide, camptothecin and teniposide, and the metal coordination complexes cisplatin and carboplatin.

Any suitable dosage may be administered in the methods of the present invention. The dosage administered will, of course, vary depending upon known factors, such as the pharmacodynamic characteristics of the particular compound and its mode and route of administration; the age, health, or weight of the subject; the nature and extent of symptoms; the metabolic characteristics of the drug and patient, the kind of concurrent treatment; the frequency of treatment; or the effect desired. Preferably, the maximum dosages administered for each drug are one half (½) the applicable LD₅₀, more preferably one third (⅓) the applicable LD₅₀, and still more preferably one fourth (¼) the applicable LD₅₀.

In one embodiment, naphthoquinones of the invention are administered at a dosage of between 0.1 mg/kg and 20 mg/kg. In a preferred embodiment, the administration of naphthoquinones is at a dosage of between 1 mg/kg and 15 mg/kg. In a more preferred embodiment, the dosage is between 5 mg/kg and 10 mg/kg.

The antiproliferative agents of the invention also may be administered within a range of suitable dosages. For example, cisplatin may be administered at a dosage between 0.2 mg/kg and 7.5 mg/kg. More preferably, cisplatin is administered at a dosage between 0.5 mg/kg and 5 mg/kg. Even more preferably, cisplatin is administered at a dosage between 1 mg/kg and 4 mg/kg.

The following examples are offered by way of illustration and not by way of limitation.

### EXAMPLES

### Example 1

The Chemopotentiation of Cisplatin by Menadione

Transplantable experimental murine fibrosarcomas (2×10⁵ RIF-1 cells) were grown intradermally in the flanks of 3 month old female C3H mice (Charles River, Holister, Calif.). When the tumors reached a volume of approximately 100 mm³, the mice were randomly assigned to each experimental group (4 mice per group).

The experimental compositions were prepared as described in Table 1.

TABLE 1

| Agent | Dose | Solvent | Supplier |
|---|---|---|---|
| Menadione | 10 mg/kg | DMSO | Sigma |
| Cisplatin | 4 mg/kg | Water for injection | David Bull Labs |

Menadione was obtained from Sigma Chemical Co. (St. Louis, Mo.) and was made to the appropriate concentration in DMSO. Cisplatin (David Bull Laboratories- Mulgrave, Australia, lot. 5201844x) was made to the appropriate concentration in water for injection. The compositions were injected systemically (i.e., intraperitoneally, i.p.), in a volume of 100 microliters. For the treatment of group 3, menadione was injected 30 minutes prior to the injection of cisplatin. After treatment, the growth of the tumors was monitored three times per week by caliper measurements of three perpendicular diameters of the tumor and calculation of tumor volume from the formula:

V=π/6×D₁×D₂×D₃,

where D₁₋₃ are the diameters in mm along the three different perpendicular axes.

It should be noted that the injected volume of drug may be altered depending on the size of animal to be injected, in order to deliver the indicated dosage. For example, injection of larger animals will require that a larger amount of drug be delivered, and consequently, may require a larger volume for injection. Appropriate concentrations of drug for delivery can be readily determined using routine methods.

The tumors were followed until they reached a size of four times their day zero treatment volume (TVQT), or up to 30 days after treatment, whichever came first. The data is expressed as the “tumor volume quadrupling time” (TVQT) mean and as the “delay.” Mean TVQT is the mean days required for individual tumors to grow to four times the tumor volume at the initial treatment day. The “delay” is the median of days required for a tumor to grow to four times the mean size of the treated group, minus the median of days required to grow to four times the mean size of the control group. The data is also expressed as the ratio of the tumor volume quadrupling time of the treated tumor over the untreated control group (TVQT/CTVQT). Increasing values of this ratio indicate increased antitumor response.

The data is presented in Table 2 below and in FIG. 3.

TABLE 2

| | | Dose | | TVQT/ | Median | Delay |
|---|---|---|---|---|---|---|
| Group | Treatment | (mg/kg) | TVQT ± S.E. | CTVQT | (TVQT) | (Days) |
| 1 | Untreated Control | — | 7.0 ± 0.4 | 1.0 | 6.9 | 0.00 |
| 2 | Menadione | 10 | 7.0 ± 0.5 | 1.0 | 7.5 | 0.61 |
| 3 | Menadione → | 10 → 4 | 11.3 ± 0.5 | 1.6 | 11.1 | 4.27 |
| | Cisplatin | | | | | |
| 4 | Cisplatin | 4 | 8.4 ± 0.3 | 1.3 | 8.3 | 1.44 |

The arrow (→) in Group 3 indicates administration 30 minutes following administration of menadione.

The results of Table 2 indicate that the antiproliferative activity of cisplatin is enhanced by the use of the chemopotentiator, menadione in that a more than additive effect was observed when both compounds were used to treat the tumor bearing mice (group 3) in comparison to the use of cisplatin alone (group 4) or menadione alone (group 2).

We claim:

1. A method of treatment of a host with a cellular proliferative disease, comprising contacting said host with a naphthoquinone and an antiproliferative agent each in an amount sufficient to modulate said cellular proliferative disease.

2. The method according to claim 1, wherein said naphthoquinone comprises menadione (Kativ-G; 2-Methyl-1,4-Naphthoquinone; Menaphthone; Vitamin K3; Panosine; 2-Methyl-1,4-naphthalenedione; Vitamin K2(0); Methyl-1,4-naphthalenedione; Methyl-1,4-naphthoquinone).

3. The method according to claim 1, wherein said naphthoquinone comprises a menadione analog.

4. The method according to claim 1 wherein said antiproliferative agent comprises an agent that interacts with nucleic acids.

5. The method according to claim 1 wherein said antiproliferative agent comprises an alkylating agent, an intercalating agent, a metal coordination complex, a pyrimidine nucleoside, a purine nucleoside, an inhibitor of nucleic acid associated enzymes, or an inhibitor of nucleic acid associated proteins.

6. The method according to claim 1 wherein said antiproliferative comprises cisplatin.

7. A method according to claim 1 wherein said naphthoquinone is administered before the administration of said antiproliferative agent.

8. A method according to claim 1 when said naphthoquinone is administered during the administration of said antiproliferative agent.

9. A method according to claim 1 when said naphthoquinone is administered after the administration of said antiproliferative agent.

10. The method of claim 1 wherein the modulation of said disease with said composition is greater than that for said antiproliferative agent alone.

11. A composition comprising a naphthoquinone and an antiproliferative agent.

12. The composition of claim 11 wherein said naphthoquinone comprises menadione.

13. Use of a naphthoquinone and an antiproliferative agent in the formulation of a medicament for the treatment of a cellular proliferative disease.
